(19) United States
(12) Patent Application Publication
(10) Pub. No.: US 2025/0172535 A1
(43) Pub. Date: May 29, 2025

(54) Method of Determining River Basin Ecological Risk with Spatial Aggregation Level and Regional Imbalance Level

(71) Applicant: Harbin Institute of Technology, Harbin City, Heilongjiang Province (CN)

(72) Inventors: Yu TIAN, Harbin (CN); Lipin LI, Harbin (CN); Weijia LI, Harbin (CN); Yedong GAO, Harbin (CN); Wei ZHAN, Harbin (CN); Nanqi REN, Harbin (CN)

(21) Appl. No.: 18/827,647

(22) Filed: Sep. 6, 2024

(30) Foreign Application Priority Data
Nov. 29, 2023 (CN) 2023116141634

(51) Int. Cl. G01N 33/24; G06F 17/11

(52) U.S. Cl. CPC G01N 33/24; G06F 17/11

(57) ABSTRACT

A method of determining ecological risk with spatial aggregation and regional imbalance level applicable for use in a river basin which involves geographic information, ecology, environmental engineering and computer technology includes the steps of: dividing a river basin into a plurality of fishnets; obtaining a basin landscape disturbance index and a basin landscape vulnerability index; obtaining a basin ecological risk index based on the basin landscape disturbance index and the basin landscape vulnerability index; and determining and evaluating the basin ecological risk in spatial aggregation and the regional imbalance with reference to a spatial autocorrelation value and a basin ecological risk imbalance value obtained by using the basin ecological risk index. The ecological risk in spatial aggregation and the regional imbalance is output and evaluate so that the ecological risk in river basin can be assessed comprehensively with high evaluation accuracy.

### CROSS REFERENCE OF RELATED APPLICATION

This is a non-provisional application that claims priority to Chinese application number 2023116141634, filing date Nov. 29, 2023, the entire contents of which are expressly incorporated herein by reference.

### BACKGROUND OF THE PRESENT INVENTION

### Field of Invention

The present invention relates to the interdisciplinary field of geographic information, ecology, environmental engineering and computer technology, in particularly related to a method of comprehensively evaluating ecological risk with spatial aggregation and regional imbalance in river basin.

### Description of Related Arts

River basin ecosystems play an important role in sustainable development by providing functions such as drinking water, industrial water, flood storage, irrigation and aquaculture. With the development of society, the development and utilization of natural resources has gradually increased, and ecological risks in river basins have become increasingly significant. The river basin is one system as a whole, and the ecological risk assessment within the river basin is one of the important contents of current ecological environment management and protection. It is of great significance to protect and maintain the stability of the river basin ecosystem and can provide a prevention and guarantee mechanism for the sustainable development of the water environment in the river basin system.

Ecological risk is the possibility of loss of system function due to changes in the composition and structure of the ecosystem, reflecting the adverse ecological effects of the ecosystem caused by human activities and changes in the natural environment. At present, traditional ecological risk assessment methods mainly include the single indicator evaluation method, the water quality and soil quality standard method, etc. However, these methods can only be used for ecological risk assessment in specific scenarios such as small-scale, single pollutant, and emergency events. However, as a complex ecosystem, the risk assessment of the water environment at the basin scale involves many factors. At the same time, under the background of continuous environmental change and regional main functional positioning, the water ecological risk in the river basin has also shown distinct regional imbalance characteristics and spatial heterogeneity in local areas. Therefore, the existing ecological risk assessment method is not suitable for basin-scale ecological risk assessment, which will lead to incomplete evaluation of the area and low accuracy of basin ecological risk assessment.

### SUMMARY OF THE PRESENT INVENTION

In order to solve the problem that the existing ecological risk assessment method is not suitable for river basin ecological risk assessment, which will lead to incomplete assessment area and low accuracy of basin ecological risk assessment,, the present invention provides a method of determining ecological risk comprehensively with spatial aggregation level and regional imbalance level in a river basin.

A method of determining ecological risk with spatial aggregation level and regional imbalance level of a river basin comprises the following steps of:
Step (a): dividing a study basin into a plurality of fishnets to obtain the river basin composed of the fishnets through GIS technology;
Step (b): obtaining a basin ecological risk index based on the river basin composed of the fishnets, specifically:
Step (b.1): obtaining a basin landscape disturbance index, which is represented by following formula:

$${D}_{i} = {dF}_{i} + {eG}_{i} + {fH}_{i}$$

$$d + e + f = 1 ,$$

wherein Dᵢ refers to a landscape disturbance index of an i-th land use type, Fᵢ refers to a landscape fragmentation level of the i-th land use type, Gᵢ refers to a landscape separation level of the i-th land use type, Hᵢ refers a fractal dimension of the i-th land use type, d refers to a landscape fragmentation coefficient, e refers to a landscape separation coefficient, f refers to a fractal dimension coefficient, and i refers to a label of the land use type;
wherein the land use type comprises a construction land, a forest land, a grassland, a cultivated land, a water area and an unused land;
Step (b.2): obtaining a basin landscape vulnerability index;
Step (b.3): obtaining a basin ecological risk index by using the basin landscape disturbance index obtained in step (b.1) and the basin landscape vulnerability index obtained in step (b.2) based on the river basin composed of the fishnets in step (a);
Step (c): evaluating and determining a basin ecological risk in spatial aggregation level and a basin ecological risk in regional imbalance level.

Furthermore, in step (a), the step of dividing the study basin into a plurality of fishnets specifically refers to: each fishnet having a square shape and an area 2˜5 times of the total area of a landscape patch.

Furthermore, the landscape fragmentation level of the i-th land use type refers to:

$${F}_{i} = \frac{{U}_{i}}{{A}_{i}}$$

where Uᵢ refers to a number of landscape patches contained in the i-th land use type, Aᵢ refers to an area of the i-th land use type;
the fractal dimension of the i-th land use type refers to:

$${H}_{i} = \frac{2 ln ( \frac{{C}_{i}}{4} )}{ln {A}_{i}}$$

where Cᵢ refers to a perimeter of the i-th land use type;
the landscape separation level of the i-th land use type refers to:

$${G}_{i} = \frac{A}{2 {A}_{i}} \sqrt{\frac{{U}_{i}}{A}}$$

where A refers a total area of all land use types.

Furthermore, in step (b.2), the landscape vulnerability index is obtained by the following process: assigning a basin landscape vulnerability index for each of the land use types, wherein the basin landscape vulnerability index of the construction land, the forest land, the grassland, the cultivated land, the water area, and the unused land increases in sequence and the unused land has the greatest basin landscape vulnerability index.

Furthermore,, in step (b.3), the basin ecological risk index is:

$${E}_{j} = {\sum }_{i = 1}^{Z} \frac{{L}_{ji}}{{L}_{j}} {D}_{i} \cdot {I}_{i}$$

where Eⱼ is a basin ecological risk index of fishnet j, Z is a number of land use types in the river basin, Lⱼᵢ is an area of a land use type i in a fishnet j, Lⱼ is a total area of the fishnet j, Dᵢ is the basin landscape disturbance index of the i-th land use type, and Iᵢ is the basin landscape vulnerability index of the i-th land use type.

Furthermore, in step (c), the basin ecological risk index obtained in step (b) is used to evaluate the basin ecological risk in spatial aggregation and the basin ecological risk in regional imbalance, specifically:
(c.1): using the basin ecological risk index obtained in step (b) to obtain a basin spatial autocorrelation value, and then using the basin spatial autocorrelation value to process assessment of the basin ecological risk in spatial aggregation and a degree of spatial difference between two adjacent fishnets through GIS technology;
the spatial autocorrelation value of the basin is:

$$M = \frac{{\sum }_{j = 1}^{n} {\sum }_{k = 1}^{n} {W}_{jk} ( {E}_{j} - \overline{E} ) ( {E}_{k} - \overline{E} )}{{S}^{2} {\sum }_{j = 1}^{n} {\sum }_{k = 1}^{n} {W}_{jk}}$$

wherein −1≤M≤1, M is a spatial autocorrelation value of the basin, n is a total number of fishnets, Wⱼₖ is an element value of the spatial weight matrix, if fishnet j and fishnet k are adjacent, then Wⱼₖ=1, if fishnet j and fishnet k are not adjacent, then Wⱼₖ=0, Eⱼ is a basin ecological risk index of fishnet j, Eₖ is a basin ecological risk index of fishnet k, E is an average value of all basin ecological risk indices, and S² is an intermediate variable;
(c.2): using the basin ecological risk index obtained in step (b) to obtain a basin ecological risk in regional imbalance value, thereby evaluating the basin ecological risk in regional imbalance through GIS technology based on the basin ecological risk in regional imbalance value.

Furthermore, the step of using the basin spatial autocorrelation value to process assessment of the basin ecological risk in spatial aggregation is specifically:
determining the basin ecological risk in spatial aggregation level based on the basin spatial autocorrelation value,
if M is greater than 0, it indicates that the ecological risk is distributed in a clustered manner; if M is less than 0, it indicates that the ecological risk is distributed dispersedly; if M is equal to 0, it indicates that the ecological risk value is randomly distributed in space;
the step of using the basin spatial autocorrelation value to process assessment of the degree of spatial difference between two adjacent fishnets is specifically:
calculating and obtaining a local autocorrelation index V:

$$V = \frac{{E}_{j} - \overline{E}}{{S}^{2}} {\sum }_{j = 1}^{n} {W}_{jk} ( {E}_{j} - \overline{E} )$$

if V is greater than 0, it means that there is a positive correlation between the two adjacent fishnets, and the spatial difference between the two adjacent fishnets is small, which is manifested as a “high-high” cluster or a “low-low” cluster; if V is less than 0, it means that there is a negative correlation between the two adjacent fishnets, and there are latent spatial outlier, which is manifested as a “high-low” cluster or a “low-high” cluster; if V equals to 0, it means that there is no correlation between the two adjacent fishnets.

Furthermore,

$${S}^{2} = \frac{1}{n} {\sum }_{j = 1}^{n} {( {E}_{j} - \overline{E} )}^{2} .$$

Furthermore, in step (c.2): the step of using the basin ecological risk index obtained in step (b) to obtain a basin ecological risk in regional imbalance value, thereby evaluating the basin ecological risk in regional imbalance level based on the basin ecological risk in regional imbalance value specifically refers to:

$${T}_{L} = {\sum }_{j = 1}^{n} [ {p}_{j} \times Log ( \frac{μ}{{E}_{j}} ) ]$$

$$μ = {\sum }_{j = 1}^{n} {p}_{j} \times {E}_{j}$$

where T_{L} is a basin ecological risk imbalance value, pⱼ is a proportion of the area of fishnet j to the total area of all fishnets, and μ is an intermediate variable,
the greater the T_{L}, the greater imbalance level of the basin ecological risk.

Furthermore, the proportion of the area of fishnet j to the total area of all fishnets pⱼ refers to:

$${p}_{j} = \frac{{L}_{j}}{{\sum }_{j = 1}^{n} {L}_{j}}$$

where Lⱼ is an area of the fishnet j.

The advantageous effect of the present invention are as follows:

The present invention provides a method for evaluating the spatial aggregation level and regional imbalance level of basin ecological risk. The present invention uses geographic spatial data as input, selects landscape ecological risk evaluation indicators, and constructs an ecological risk comprehensive evaluation model from three aspects: the degree of disturbance of the basin ecosystem, the sensitivity of land use, and the imbalance of spatial distribution. The evaluation results are visualized using remote sensing and GIS technology, and the ecological risk status and spatial distribution characteristics of the basin to be evaluated are obtained. The core beneficial effects of the present invention are mainly reflected in the following five points:

(1) The present invention utilizes landscape patches as the basic component unit of risk assessment and basin fishnets as the basic calculation unit, and adopts ecological risk assessment indicators such as landscape fragmentation, landscape separation, fractal dimension and landscape vulnerability index to reflect the impact of land use type changes in the river basin on the water ecosystem from different perspective. By weighting and summing these indicators, the ecological risk value is obtained, which improves the accuracy of water ecological risk assessment at the basin scale.

(2) The present invention uses geospatial data as input and uses remote sensing and GIS technology for data processing and analysis, which reduces the reliance on statistical data, avoids the data missing and inaccurate data problems in traditional evaluation methods, therefore improves the data quality and accessibility of risk assessment, and realizes the procedural and convenient calculation of ecological risk assessment at the basin scale.

(3) The present invention uses remote sensing and GIS technology to visualize the evaluation results, generate basin-scale ecological risk regional distribution maps, spatial autocorrelation distribution maps, and regional imbalance distribution maps, which can intuitively display the ecological risk status and spatial distribution characteristics of each region in the basin, improve the visualization and comprehensibility of the evaluation, and make the evaluation area more comprehensive.

(4) The present invention combines basin ecological risk assessment with spatial autocorrelation analysis, utilizes the rich information of spatial data and the powerful function of spatial analysis, comprehensively considers the multidimensional characteristics and multi-level influencing factors of the ecosystem, can fully display the heterogeneity and complexity of data, avoids ignoring or underestimating the degree of ecological risk and the scope of impact in certain areas, identifies high-high aggregation areas, low-low aggregation areas, high-low dislocation areas and low-high dislocation areas of ecological risks, and improves the comprehensiveness, accuracy, applicability and pertinence of spatial aggregation evaluation of basin ecological risks.

(5) The present invention combines the basin ecological risk assessment with the Theil's L index regional imbalance analysis, which can decompose the ecological risk differences within and between regions, accurately reflect the spatial distribution characteristics of ecological risks between regions in the basin, and improve the accuracy of the assessment of imbalance in water ecological risk areas in the basin.

### BRIEF DESCRIPTION OF THE DRAWINGS

FIG. 1 is a schematic flowchart of the present invention.

FIG. 2A is an exemplary ecological risk regional distribution map generated by the method of the present invention.

FIG. 2B is an exemplary spatial autocorrelation distribution map generated by the method of the present invention.

FIG. 2C is an exemplary regional imbalance distribution map generated by the method of the present invention.

### DETAILED DESCRIPTION OF THE PREFERRED EMBODIMENT

In order to explain further the technical problems, the technical solutions and the advantageous effects of the present invention, the present invention is further described in details below with reference to the drawings and embodiments. It should be understood that the specific embodiments described here are only used to explain the present invention and are not intended to be limiting.

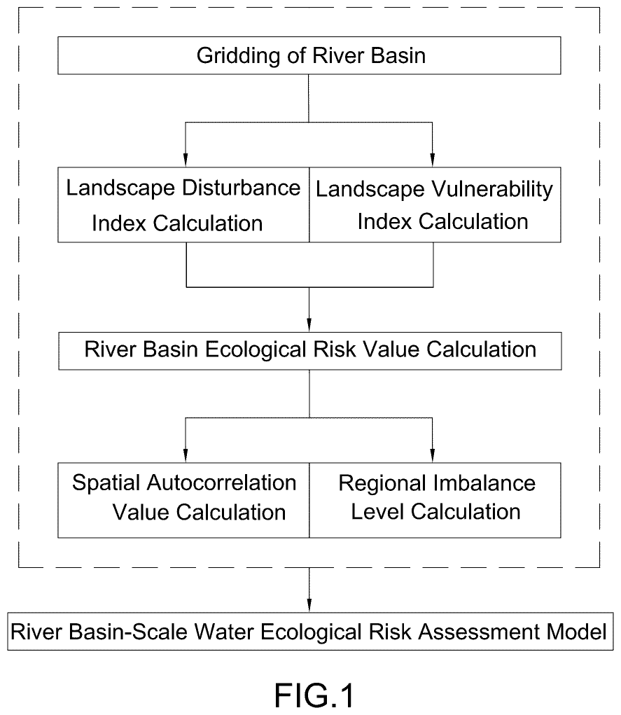

Embodiment 1: Referring to FIG. 1 of the drawing, the specific steps of the method of determining basin ecological risk with spatial aggregation and regional imbalance assessment, with at least one step executed through a processor of a computer, are as follows:

Step (a): Select a river basin and dividing the river basin into a plurality of fishnets through GIS technology to obtain the river basin with divided fishnets.

Based on an area distribution of landscape patches in the river basin, an area of one single fishnet is 2˜5 times of a total area of a landscape patch. The fishnet is a square having a dimension of N (km)×N (km). Process spatialization of the ecological risk index of the basin landscape.

Step (b): Obtain a basin ecological risk index based on the river basin with divided fishnets, which includes the following steps:
Step (b.1): Obtain Basin Landscape Disturbance Index:

The landscape disturbance index reflects the degree of external disturbance to the ecosystem represented by different landscapes. The greater the disturbance to the region, the greater the ecological risk. The landscape disturbance index is calculated by combining three indices: the landscape fragmentation level, the landscape separation level, and the fractal dimension. The landscape fragmentation level represents the degree of fragmentation of the landscape and reflects the complexity of the landscape spatial structure. The landscape separation level refers to the degree of separation of individual distribution of different patches in a certain land use type. The Fractal dimension is an indicator that quantifies complexity as the ratio of detail variation to scale variation to characterize fractal patterns or ensembles. It is used to measure the impact of patch shape on internal patch ecological processes.

The basin landscape disturbance index refers to:

$$\begin{matrix}{D}_{i} = {dF}_{i} + e {G}_{i} + f {H}_{i} \\ {F}_{i} = \frac{{U}_{i}}{{A}_{i}} \\ {G}_{i} = \frac{A}{2 {A}_{i}} \sqrt{\frac{{U}_{i}}{A}} \\ {H}_{i} = \frac{2 ln ( \frac{{C}_{i}}{4} )}{ln {A}_{i}} \\ d + e + f = 1\end{matrix}$$

wherein Dᵢ refers to a landscape disturbance index of an i-th land use type, Fᵢ refers to a landscape fragmentation level of the i-th land use type, Gᵢ refers to a landscape separation level of the i-th land use type, Hᵢ refers a fractal dimension of the i-th land use type, d refers to a landscape fragmentation coefficient, e refers to a landscape separation coefficient, f refers to a fractal dimension coefficient, Uᵢ refers to a number of landscape patches contained in the i-th land use type, Aᵢ refers to an area of the i-th land use type, in km², A refers a total area of all land use types, Cᵢ refers to a perimeter of the i-th land use type, and i refers to a label of the land use type.

The land use types include: construction land, forest land, grassland, cultivated land, water area and unused land.

In this step, d, e, and f are set to 0.5, 0.3, and 0.2, respectively.
Step (b.2): Obtain Basin Landscape Vulnerability Index:

The landscape vulnerability index reflects the sensitivity of land use types to disturbance factors. The larger the vulnerability index, the more unstable the landscape. The landscape vulnerability index generally assigns weight values to different land use types based on expert experience and literature data. The landscape vulnerability index of the present invention allocates weight values to different land use types according to literature data. Unused land is the most vulnerable, followed by water area, and construction land is the most stable. Therefore, after grading and normalization, the vulnerability index of a specific type of landscape is obtained. The landscape vulnerability classification of different land use types is shown in Table 1.

TABLE 1
Landscape Vulnerability Classification
of Different Land Use Types

| Land Use Type | Vulnerability Classification |
|---|---|
| Construction land | 1 |
| Forest land | 2 |
| Grassland | 3 |
| Cultivated land, | 4 |
| Water area | 5 |
| Unused land | 6 |

Step (b.3): Based on the river basin with divided fishnets in step (a), utilize the basin landscape disturbance index obtained in step (b.1) and the basin landscape vulnerability index obtained in step (b.2) to obtain the basin ecological risk index, specifically:

The ecological risk index represents a region's ability to resist external interference. The larger the regional ecological risk value, the weaker the resistance to interference factors. The basin ecological risk index is calculated by combining the landscape disturbance index and the landscape vulnerability index, which can comprehensively reflect the disturbance information of natural and human factors on the river basin ecology. The calculation method is shown in the following formula:

$${E}_{j} = {\sum }_{i = 1}^{Z} \frac{{L}_{ji}}{{L}_{j}} {D}_{i} \cdot {I}_{i}$$

where Eⱼ is a basin ecological risk index of fishnet j, Z is a number of land use types in the river basin, Lⱼᵢ is an area of a land use type i in a fishnet j, km², Lⱼ is a total area of the fishnet j, km², Dᵢ is the basin landscape disturbance index of the i-th land use type, and Iᵢ is the basin landscape vulnerability index of the i-th land use type.

After calculating the ecological risk value, the spatial distribution of the risk value is obtained by the empirical Bayesian Kriging method, and the pyramid classification method is used to complete the classification of low ecological risk, medium ecological risk, high ecological risk and severe ecological risk. The result can be output and displayed as an ecological risk regional distribution map.

Step (c): The basin ecological risk index obtained in step (b) is used to evaluate basin ecological risks in spatial aggregation and regional imbalance, including the following steps:
Step (c.1): The basin ecological risk index obtained in step (b) is used to obtain the basin spatial autocorrelation value, thereby evaluating the spatial aggregation of ecological risks through GIS technology. Specifically:

First, spatial autocorrelation reflects the spatial characteristics of ecological risk values in a river basin by detecting the correlation between the same feature values in different spatial units. The global spatial autocorrelation is described by Moran's I index, which reflects the trend of discreteness or aggregation of the overall spatial data in the basin as well as the intensity and significance of this trend. The spatial autocorrelation value of the basin is:

$$\begin{matrix}M = \frac{{\sum }_{j = 1}^{n} {\sum }_{k = 1}^{n} {W}_{jk} ( {E}_{j} - \overline{E} ) ( {E}_{k} - \overline{E} )}{{S}^{2} {\sum }_{j = 1}^{n} {\sum }_{k = 1}^{n} {W}_{jk}} \\ {S}^{2} = \frac{1}{n} {\sum }_{j = 1}^{n} {( {E}_{j} - \overline{E} )}^{2}\end{matrix}$$

wherein −1≤M≤1, M is the spatial autocorrelation value of the basin, n is the total number of fishnets, Wⱼₖ is the element value of the spatial weight matrix, if fishnet j and fishnet k are adjacent, then Wⱼₖ=1, if fishnet j and fishnet k are not adjacent, then Wⱼₖ=0, Eⱼ is the ecological risk index of fishnet j, Eₖ is the ecological risk index of fishnet k, Ē is the average of all ecological risk indices, and S² is an intermediate variable.

Then, the spatial aggregation of the ecological risk in the current basin is determined according to the size of the basin spatial autocorrelation value.

The range of M is −1 to 1. When M is greater than 0, it indicates that the ecological risk is distributed in a clustered manner; when M is less than 0, it indicates that the ecological risk is distributed dispersedly; when M is equal to 0, it indicates that the ecological risk value is randomly distributed in space.

Finally, the spatial differences between adjacent fishnets are evaluated according to the basin ecological risk index, specifically:

Since global spatial autocorrelation considers the entire river basin, spatial outliers cannot be found. Therefore, local autocorrelation is used to analyze the spatial difference and significance of ecological risk values in each fishnet and its adjacent fishnets. The calculation method is shown in the following formula:

$$V = \frac{{E}_{j} - \overline{E}}{{S}^{2}} {\sum }_{j = 1}^{n} {W}_{j k} ( {E}_{j} - \overline{E} )$$

where V is the local autocorrelation index. When V is greater than 0, it means that there is a positive correlation between the fishnet and its adjacent fishnet, and the spatial difference between adjacent fishnet is small, which is manifested as a “high-high” cluster or a “low-low” cluster. When V is less than 0, it means that there is a negative correlation between the fishnet and its adjacent fishnet, and there are potential spatial outlier, which are manifested as a “high-low” cluster or a “low-high” cluster. When V equals to 0, it means that there is no correlation between the fishnet and its adjacent fishnet.
Step (c.2): The basin ecological risk index obtained in step (b) is used to obtain the imbalance value of the basin ecological risk region, specifically:

Based on the calculation results of the ecological risk index, the weighted Theil's L index is used to evaluate the regional differences and measure the imbalance of ecological risks among regions at the basin scale. The larger the value, the more unbalanced the ecological risks among regions in the basin. The calculation process is as follows:
where T_{L} is an indicator of ecological risk imbalance in the basin, Lⱼ is an area of the fishnet j, pⱼ is the proportion of the area of fishnet j to the total area of all fishnets, and μ is the intermediate variable.

The data required for all the above calculation processes come from the Resource and Environmental Science and Data Center of the Chinese Academy of Sciences (https://www.resdc.cn/Default.aspx), and the collected data type is land use type data. Some parameters are determined based on the conclusions of relevant studies. These parameters can be comprehensively determined based on local actual conditions and relevant research results. The regional imbalance analysis is processed through GIS technology. The results may be output and displayed as a spatial autocorrelation distribution map and a regional imbalance distribution map.

Exemplary Embodiment: In order to verify the beneficial effects of the present invention, the present invention is applied in the assessment of ecological risks in the Songhua River Basin and the result is successful, specifically:

The Songhua River is one of the seven major rivers in China. With the development and utilization of coastal land and changes in land use patterns, the ecological and environmental security of the basin is threatened, and obvious ecological and environmental problems such as wetland shrinkage, land desertification, and salinization have emerged. The Songhua River Basin is an important grain base in China, and its ecological and environmental security is of vital importance. Therefore, in order to promote the ecological protection of the Songhua River Basin and promote the coordinated and sustainable development of the basin, it is necessary to comprehensively measure the spatial distribution characteristics of water ecological risks in the Songhua River Basin.

Landscape patch is a concept in landscape ecology, which refers to a relatively homogeneous nonlinear area that is different from the surrounding background. It is the basic component unit of landscape pattern. Landscape patches can be naturally formed, such as forests, lakes or grasslands, or artificially created, such as farmland or urban parks.

The specific implementation process is as follows:

### 1. Dividing into Fishnets

According to an area distribution of landscape patches in the Songhua River Basin, dividing the basin into a plurality of fishnets. An area of one single fishnet is 2˜5 times an area of the landscape patch. Since it is a case study serving as an example only, in order to speed up the calculation, this example divides the basin into a plurality of square fishnets with a side length of 20 km×20 km. The fishnet division process is completed in ArcGIS software, with a total number of 1528 fishnets.

### 2. Processing Calculation of Basin Landscape Disturbance Index

Import the Songhua River Basin which are divided into the fishnets into the Fragstats software, calculate a patch area of each fishnet, a number of patch area (Uᵢ), an area of each land use type (Aᵢ), an area of all land use type (A), a perimeter of each land use type (Cᵢ). Process calculations based on the above Embodiment, landscape disturbance index (Dᵢ), landscape fragmentation level (Fᵢ), landscape separation level (Gᵢ) and fractal dimension (Hᵢ) are obtained.

According to this example, assign 0.5, 0.3 and 0.2 to d, e, and f respectively. According to the data source, the land use type are divided into six primary categories, namely construction land, forest land, grassland, cultivated land, water area and unused land.

### 3. Processing Calculation of Basin Landscape Vulnerability Index

The landscape vulnerability index assigns weight values to different land use types based on literature data. Unused land is the most vulnerable, followed by water areas, and construction land is the most stable. After classification and normalization, the vulnerability index of a specific type of landscape is obtained, which as shown in Table 2.

TABLE 2
Vulnerability Classification of Different Land Use Types

| Land Use Type | Vulnerability Classification | Vulnerability Index |
|---|---|---|
| Construction land | 1 | 0.05 |
| Forest land | 2 | 0.10 |
| Grassland | 3 | 0.14 |
| Cultivated land, | 4 | 0.19 |
| Water area | 5 | 0.24 |
| Unused land | 6 | 0.29 |

### 4. Processing Calculation of Basin Ecological Risk Index

The above process can obtain the landscape disturbance index (Dᵢ) and landscape vulnerability index (Iᵢ) of each fishnet, thereby the ecological risk index (Eⱼ) of each fishnet is obtained.

After calculating the ecological risk value of each fishnet, the results are linked to the AcrGIS software. The spatial distribution of ecological risk values in the Songhua River Basin is obtained by the empirical Bayesian Kriging method, and the low ecological risk, medium ecological risk, high ecological risk and severe ecological risk levels are divided using the pyramid classification method. Combined with comprehensive analysis of land use type data, it is found that severe risk areas are mainly distributed in the water areas and the unused lands, high risk areas are mainly distributed in some of the cultivated lands, the water areas and the unused lands, and medium risk and low risk areas are mainly distributed in the cultivated land, the grassland and the forest land.

### 5. Processing Calculation of Spatial Autocorrelation

The global spatial autocorrelation and local spatial autocorrelation are calculated by ArcGIS. The global spatial autocorrelation is 0.68, and the z score is 50.98, indicating that the probability of randomly generating this clustering pattern is less than 1%, and the ecological risk in the Songhua River Basin has a significant spatial positive correlation. Therefore, it can be seen that the high-value clusters of ecological risk values in the Songhua River Basin are mainly distributed in high-risk areas, namely some cultivated land, water areas and unused land, which may be due to the relatively high intensity of interference from human activities; and the low-value clusters are mainly distributed in the forest land, indicating that the ecological environment quality is good.

### 6. Processing Calculation of Imbalance of Basin Ecological Risk

The Theil's L index of the basin calculated based on the ecological risk index of each fishnet is 0.07. This value is close to 0, indicating that the overall ecological risk distribution in the Songhua River Basin is relatively balanced. Low level imbalance is shown in areas with higher ecological risks, while relatively higher imbalance of the ecological risk is shown in forest land and grassland, indicating that although the ecological risk values of forest land and grassland are low, they still need to be paid attention to.

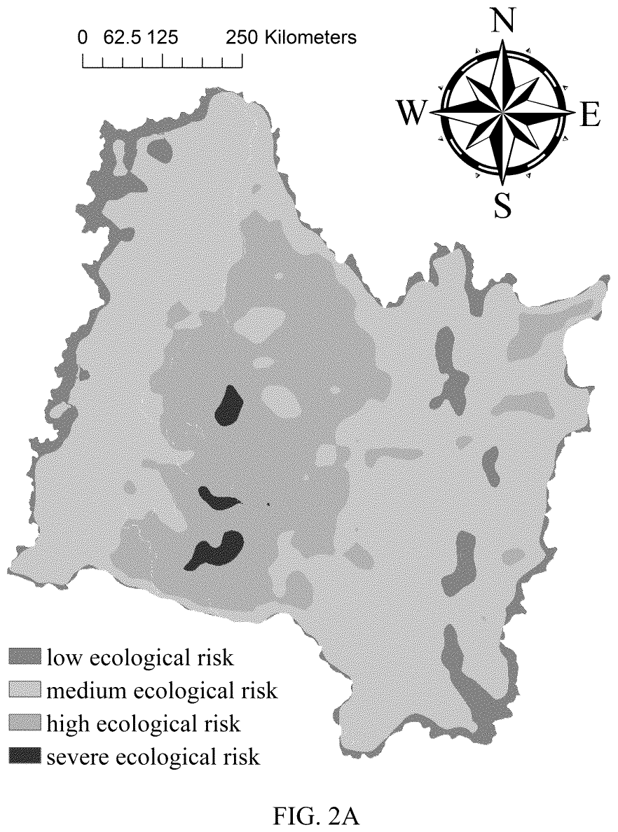

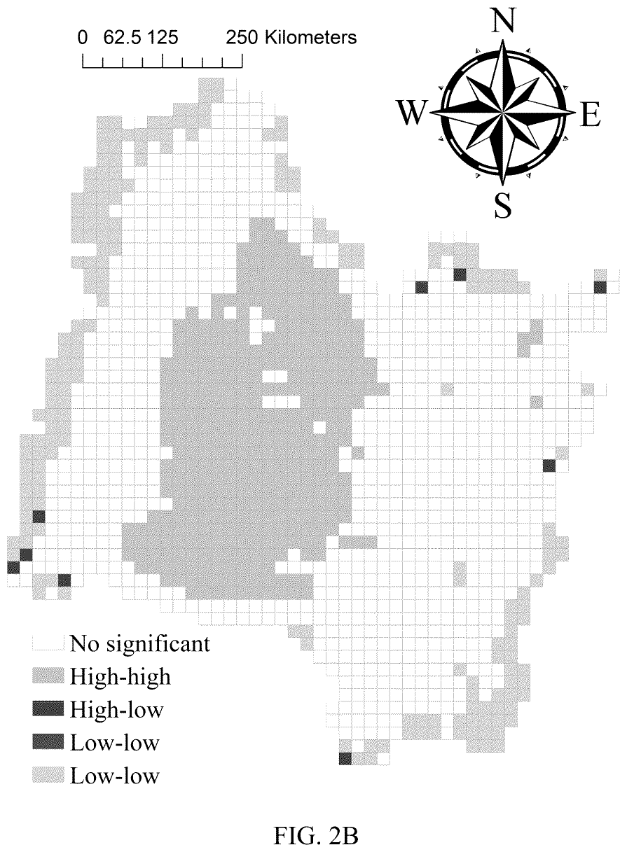

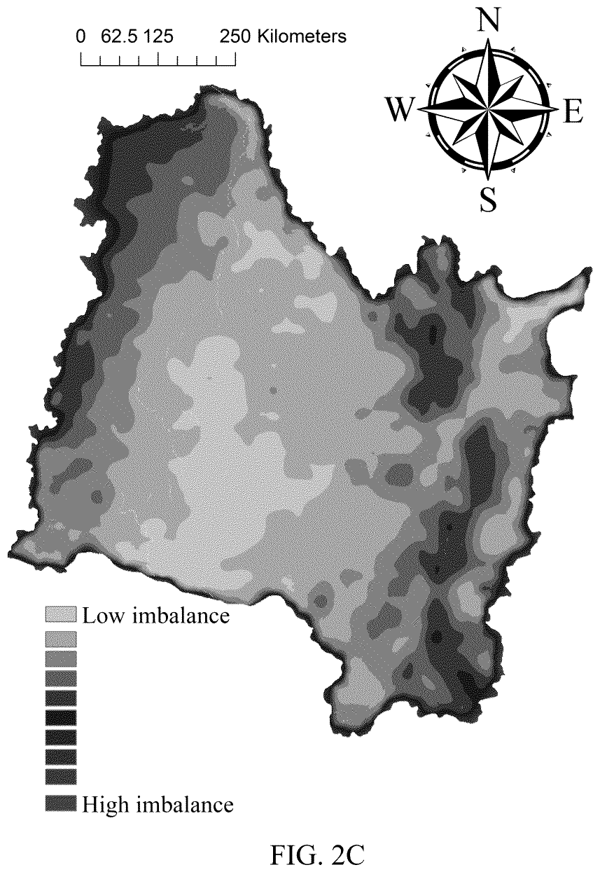

The exemplary ecological risk regional distribution map, spatial autocorrelation distribution map, and regional imbalance distribution map generated by the method of the present invention are shown in FIG. 2A, FIG. 2B and FIG. 2C.The basic principles and main features of the present invention and the advantages of the present invention have been shown and described above. Those skilled in the art should understand that the present invention is not limited by the above embodiments. The above embodiments and descriptions only illustrate the principles of the present invention. Without departing from the spirit and scope of the present invention, there will be various changes and improvements in the present invention, and these changes and improvements will all fall within the scope of the claimed invention. The scope of protection of the present invention is defined by the appended claims and their equivalents.

What is claimed is:

1. A method of determining ecological risk with spatial aggregation level and regional imbalance level of a river basin, characterized in that, the method comprising the steps of:
(a): dividing the river basin into a plurality of fishnets to obtain the river basin composed of the fishnets;
(b) obtaining a basin ecological risk index based on the river basin composed of the fishnets by the sub-steps of:
(b.1): obtaining a basin landscape disturbance index, which is represented by formula:
$$\begin{matrix}{D}_{i} = d {F}_{i} + e {G}_{i} + f {H}_{i} \\ d + e + f = 1 ,\end{matrix}$$
where Dᵢ refers to a landscape disturbance index of an i-th land use type, Fᵢ refers to a landscape fragmentation level of the i-th land use type, Gᵢ refers to a landscape separation level of the i-th land use type, Hᵢ refers a fractal dimension of the i-th land use type, d refers to a landscape fragmentation coefficient, e refers to a landscape separation coefficient, f refers to a fractal dimension coefficient, and i refers to a label of the land use type;
wherein the land use type comprises a construction land, a forest land, a grassland, a cultivated land, a water area and an unused land;
(b.2): obtaining a basin landscape vulnerability index;
(b.3) obtaining a basin ecological risk index by using the basin landscape disturbance index obtained in step (b.1) and the basin landscape vulnerability index obtained in step (b.2) based on the river basin composed of the fishnets in step (a);
(c) determining and evaluating a basin ecological risk in spatial aggregation level and a basin ecological risk in regional imbalance level.

2. The method of determining ecological risk with spatial aggregation and regional imbalance of a river basin according to claim 1, characterized in that, each of the fishnets is a square fishnet having an area 2˜5 times of a total area of a landscape patch.

3. The method of determining ecological risk with spatial aggregation and regional imbalance of a river basin according to claim 2, characterized in that,
a landscape fragmentation level of the i-th land use type is:
$${F}_{i} = \frac{{U}_{i}}{{A}_{i}}$$
where Uᵢ refers to a number of landscape patches contained in the i-th land use type, Aᵢ refers to an area of the i-th land use type;
a fractal dimension of the i-th land use type is:
$${H}_{i} = \frac{2 ln ( \frac{{C}_{i}}{4} )}{ln {A}_{i}}$$
where Cᵢ refers to a perimeter of the i-th land use type;
a landscape separation level of the i-th land use type is:
$${G}_{i} = \frac{A}{2 {A}_{i}} \sqrt{\frac{{U}_{i}}{A}}$$
where A refers a total area of all land use types.

4. The method of determining ecological risk with spatial aggregation and regional imbalance of a river basin according to claim 3, characterized in that, in step (b.2), the landscape vulnerability index is obtained by: assigning a basin landscape vulnerability index for each of the land use types, wherein the basin landscape vulnerability index of the construction land, the forest land, the grassland, the cultivated land, the water area, and the unused land increases in sequence and the unused land has the greatest basin landscape vulnerability index.

5. The method of determining ecological risk with spatial aggregation and regional imbalance of a river basin according to claim 4, characterized in that, in step (b.3), the basin ecological risk index is:
$${E}_{j} = {\sum }_{i = 1}^{Z} \frac{{L}_{ji}}{{L}_{j}} {D}_{i} \cdot {I}_{i}$$
where Eⱼ is a basin ecological risk index of fishnet j, Z is a number of land use types in the river basin, Lⱼᵢ is an area of a land use type i in a fishnet j, Lⱼ is a total area of the fishnet j, Dᵢ is the basin landscape disturbance index of the i-th land use type, and It is the basin landscape vulnerability index of the i-th land use type.

6. The method of determining ecological risk with spatial aggregation and regional imbalance of a river basin according to claim 5, characterized in that, in step (c), the basin ecological risk index obtained in step (b) is used to evaluate the basin ecological risk in spatial aggregation and the basin ecological risk in regional imbalance, specifically:
(c.1): using the basin ecological risk index obtained in step (b) to obtain a basin spatial autocorrelation value, and then using the basin spatial autocorrelation value to process assessment of the basin ecological risk in spatial aggregation and a degree of spatial difference between two adjacent fishnets;
the spatial autocorrelation value of the basin is:
$$M = \frac{{\sum }_{j = 1}^{n} {\sum }_{k = 1}^{n} {W}_{jk} ( {E}_{j} - \overline{E} ) ( {E}_{k} - \overline{E} )}{{S}^{2} {\sum }_{j = 1}^{n} {\sum }_{k = 1}^{n} {W}_{jk}}$$
wherein −1≤M≤1, M is a spatial autocorrelation value of the basin, n is a total number of fishnets, Wⱼₖ is an element value of the spatial weight matrix, if fishnet j and fishnet k are adjacent, then Wⱼₖ=1, if fishnet j and fishnet k are not adjacent, then Wⱼₖ=0, Eⱼ is a basin ecological risk index of fishnet j, Eₖ is a basin ecological risk index of fishnet k, Ē is an average value of all basin ecological risk indices, and S² is an intermediate variable;
(c.2): using the basin ecological risk index obtained in step (b) to obtain a basin ecological risk in regional imbalance value, thereby evaluating the basin ecological risk in regional imbalance based on the basin ecological risk in regional imbalance value.

7. The method of determining ecological risk with spatial aggregation and regional imbalance of a river basin according to claim 6, characterized in that, the step of using the basin spatial autocorrelation value to process assessment of the basin ecological risk in spatial aggregation refers to:
determining the basin ecological risk in spatial aggregation based on the basin spatial autocorrelation value,
if M is greater than 0, it indicates that the ecological risk is distributed in a clustered manner; if M is less than 0, it indicates that the ecological risk is distributed dispersedly; if M is equal to 0, it indicates that the ecological risk value is randomly distributed in space;
the step of using the basin spatial autocorrelation value to process assessment of the degree of spatial difference between two adjacent fishnets refers to:
calculating and obtaining a local autocorrelation index V:
$$V = \frac{{E}_{j} - \overline{E}}{{S}^{2}} {\sum }_{j = 1}^{n} {W}_{j k} ( {E}_{j} - \overline{E} )$$
if V is greater than 0, it means that there is a positive correlation between the two adjacent fishnets, and the spatial difference between the two adjacent fishnets is small, which is manifested as a “high-high” cluster or a “low-low” cluster; if V is less than 0, it means that there is a negative correlation between the two adjacent fishnets, and there are latent spatial outlier, which is manifested as a “high-low” cluster or a “low-high” cluster; if V equals to 0, it means that there is no correlation between the two adjacent fishnets.

8. The method of determining ecological risk with spatial aggregation and regional imbalance of a river basin according to claim 7, characterized in that,
$${S}^{2} = \frac{1}{n} {\sum }_{j = 1}^{n} {( {E}_{j} - \overline{E} )}^{2} .$$

9. The method of determining ecological risk with spatial aggregation and regional imbalance of a river basin according to claim 8, characterized in that, in step (c.2): the step of using the basin ecological risk index obtained in step (b) to obtain a basin ecological risk in regional imbalance value, thereby evaluating the basin ecological risk in regional imbalance based on the basin ecological risk in regional imbalance value specifically refers to:
$$\begin{matrix}{T}_{L} = {\sum }_{j = 1}^{n} [ {p}_{j} \times Log ( \frac{μ}{{E}_{j}} ) ] \\ μ = {\sum }_{j = 1}^{n} {p}_{j} \times {E}_{j}\end{matrix}$$
where T_{L} is a basin ecological risk imbalance value, pⱼ is a proportion of the area of fishnet j to the total area of all fishnets, and μ is an intermediate variable;
the greater the T_{L}, the more unbalanced the basin ecological risk.

10. The method of determining ecological risk with spatial aggregation and regional imbalance of a river basin according to claim 9, characterized in that, the proportion of the area of fishnet j to the total area of all fishnets pⱼ refers to:
$${p}_{j} = \frac{{L}_{j}}{{\sum }_{j = 1}^{n} {L}_{j}}$$
where Lⱼ is an area of the fishnet j.
